(19) United States
(12) Patent Application Publication
(10) Pub. No.: US 2025/0001423 A1
(43) Pub. Date: Jan. 2, 2025

(54) POLYMERASE CHAIN REACTION WELL INCLUDING A SIDE WALL WITH A FLUOROPOLYMER

(71) Applicant: Hewlett-Packard Development Company, L.P., Spring, TX (US)

(72) Inventor: Rajesh Kelekar, Palo Alto, CA (US)

(73) Assignee: Hewlett-Packard Development Company, L.P., Spring, TX (US)

(21) Appl. No.: 18/292,849

(22) PCT Filed: Aug. 5, 2021
(86) PCT No.: PCT/US2021/044763

§ 371 Date: Jan. 26, 2024

(51) Int. Cl. B01L 7/00; C12Q 1/686; G01N 21/64

(52) U.S. Cl. CPC B01L 7/52; C12Q 1/686; G01N 21/6486; B01L 2200/0668; B01L 2300/0663; B01L 2300/0829; B01L 2300/16; B01L 2300/1827; B01L 2400/043

(57) ABSTRACT

A device includes at least one well to receive a polymerase chain reaction (PCR) mixture with the at least one well including a bottom and side walls. The bottom includes an electrically resistive sheet including at least one opening and to receive a signal from a signal source to generate heat to cause a pulse-controlled amplification, thermal cycling zone in close thermal proximity to the resistive sheet. The side walls comprise a polymer material and a fluoropolymer component to cause the PCR mixture to form a contact angle of at least 45 degrees relative to the side walls. An optical detector is alignable with the at least one opening to detect fluorophores as an output of the PCR mixture.

### BACKGROUND

Molecular diagnostics has revolutionized modern medicine. Some types of such diagnostics may employ polymerase chain reaction (PCR) processes to rapidly make many copies of partial DNA strands.

### BRIEF DESCRIPTION OF THE DRAWINGS

Each of FIGS. 1A and 1B are a sectional side view of an example testing device including an example well to receive a polymerase chain reaction mixture.

FIG. 2 is a block diagram of an example control portion.

FIG. 3 is an isometric view of an example testing device including multiple PCR wells.

Each of FIGS. 5 and 6 are a flow diagram of an example method of forming a PCR well.

FIG. 6 is a block diagram schematically representing an example formation engine.

FIG. 7A is a block diagram schematically representing an example operations engine.

FIGS. 7B and 7C are each a block diagram schematically representing an example control portion and an example user interface, respectively.

FIG. 8 is a flow diagram of an example method of testing including performing a polymer chain reaction (PCR).

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to a side wall of a PCR well arranged to reduce the wetting of a polymerase chain reaction (PCR) mixture relative to the side walls. In some examples, the PCR well forms part of a testing device to perform pulse-controlled amplification (PCA), polymerase chain reactions. However, in some examples, the PCR well of the testing device may be used to perform other molecular testing and the testing device may sometimes be referred to as a molecular testing device.

In some examples, the testing device may comprise at least one well to receive a polymer chain reaction (PCR) mixture with the at least one well including a bottom and side walls. The side walls may comprise a polymer material and a fluoropolymer component to cause a contact angle between the side wall and the PCR mixture such that a non-wetting or reduced wetting behavior is exhibited at the side wall. In some such examples, the contact angle may comprise at least 45 degrees.

In some examples, via reduced wetting properties due to the fluoropolymer component of the side walls, the reaction volume may sometimes be referred to as exhibiting a generally uniform distribution within the well at least in the sense that reaction volume has a height which is substantially the same across a width of the PCR well.

In some examples, the desired effects from providing side walls having a reduced wetting properties also may be achieved according to a contact angle which produces a minimally concave meniscus at least because a greater proportion of the reaction volume is located in more central area of the PCR well even though a flat or convex meniscus was not achieved. In some examples, a minimally concave meniscus (or generally uniform distribution of the reaction volume) may be expressed as a height of the reaction volume within, and across a width of the well, having a variance of less than about 10 percent or less than about 5 percent in some examples.

In some examples, a device includes at least one well to receive a polymerase chain reaction (PCR) mixture with the at least one well including a bottom and side walls. The bottom includes an electrically resistive sheet including at least one opening and to receive a signal from a signal source to generate heat to cause a pulse-controlled amplification, thermal cycling zone in close thermal proximity to the resistive sheet. In some examples, the side walls comprise a polymer material and a fluoropolymer component to cause an overall volume of the PCR mixture to form a contact angle of at least 45 degrees relative to the side walls. An optical detector is alignable with the at least one opening to detect fluorophores as an output of the PCR mixture.

In some examples, the PCR mixture may comprise superparamagnetic beads functionalized with single-stranded nucleic acids (e.g. DNA strands, RNA strands) within the PCR mixture to facilitate guiding the nucleic acid strands into the thermal cycling zone within the well. Accordingly, in some examples, the device may comprise a magnet (e.g. external magnet) to apply an external magnetic force to attract the superparamagnetic beads (and associated single-strands of nucleic acids) into the thermal cycling zone.

In some such examples, by arranging the side walls of the PCR well to include a fluoropolymer component which causes a contact angle (e.g. at least 45 degrees), one may achieve a target meniscus having a shape (e.g. flat, convex, minimally concave) such that the overall volume of the PCR mixture exhibits a generally uniform distribution across a width of the interior of the PCR well. By doing so, a greater proportion of the PCR volume is present within the thermal cycling zone at which the pulse-controlled amplification is to occur upon generation of heat via the electrically resistive sheet. Among other aspects, this arrangement may increase the efficacy of testing because a greater proportion of the PCR volume may complete the polymerase chain reaction. In addition, the more uniform distribution of the overall volume within the PCR well also enables a greater proportion of output elements (e.g. fluorophores) of the polymerase chain reaction to migrate or diffuse (within the at least one well) into alignment with the optical detector. This arrangement may increase the accuracy and/or sensitivity of the testing. Furthermore, the more uniform distribution of the overall volume of the PCR mixture within the at least one well (due to the reduced wetting properties along the side wall) also enables a greater proportion of superparamagnetic beads (functionalized with single-stranded nucleic acids) to become aligned with the externally applied magnetic force, which in turn causes a greater proportion of the beads (and therefore the associated single-stranded nucleic acids) to be pulled into the thermal cycling zone for pulse-controlled amplification to facilitate the polymerase chain reaction.

Whether viewed separately or together, these effects of reducing the wetting properties of the side wall may enhance a pulse-controlled amplification, polymerase chain reaction or other molecular testing.

The example arrangements (including a fluoropolymer component as part of the side walls of a PCR well) produce a structure which is robust and less susceptible to cracking (or other deterioration) such as plasma treatments and/or other materials such as silane and which exhibits desired reduced wetting properties which may not be achievable via the plasma treatments and/or silane materials.

These examples, and additional examples, are described below in association with at least FIGS. 1A-8.

FIG. 1A is side sectional view of a testing device 100 comprising an example well 105. As shown in FIG. 1A, the well 105 comprises a bottom 120 and side wall(s) 110 extending vertically upward from the bottom 120. The bottom comprises a first surface (e.g. external surface) 117B and a second surface (e.g. internal surface) 117A, while each side wall 110 comprises an external surface 113 and internal surface 114. Together, the side wall(s) 110 and bottom 120 define an interior 125 of the well 105, which defines a receptacle to receive a polymerase chain reaction (PCR) mixture 240 (FIG. 1B). Accordingly, the well 105 may sometimes be referred to as a PCR well 105. In some examples, the PCR mixture comprises a PCR mixture suitable for performing pulse-controlled amplification (PCA)-type polymerase chain reactions. Accordingly, the PCR mixture may sometimes be referred to as a PCA mixture or PCA-PCR mixture. In some examples, overall volume of the PCR mixture 240 received into the well 105 may comprise between about 40 microliters to about 50 microliters.

At least the inner surface 114 of side walls 110 and the first surface 117A of bottom 120 comprise, and/or are coated with, an inert material so as to not affect the PCR mixture 240 and related reaction processes.

Moreover, in some examples, the internal surface 114 of the side wall 110 comprises a reduced-wetting portion 116, which may comprise a coating or other structure to exhibit the reduced-wetting properties and which may be inert to the PCR mixture 240. In some examples, the coating may comprise a nanocoating.

In some examples, the electrically resistive sheet 121 of bottom 120 comprises a portion 130 including a plurality of openings 132 though which light may be transmitted to enable optical detection of output elements resulting from the PCA-type, polymerase chain reaction. At least some example output elements comprise fluorophores, which may be represented by reference numerals F, as later shown in FIG. 1B. While not shown for illustrative clarity, the bottom 120 may further comprise a carrier layer secured to the second surface 117B of the resistive sheet 121 with the carrier layer being transparent in at least the regions corresponding to the location of the openings 132 of the resistive sheet 121.

In some examples, the PCR well 105, 205 shown in FIGS. 1A-1B may include a lid or cover comprising transparent materials, which may comprise materials similar to those identified herein for forming, constructing second element 123.

FIG. 1B is side sectional view schematically representing a testing device 200 including an example PCR well 205. In some examples, the device 200 may comprise at least some of substantially the same features and attributes as the device 100 of FIG. 1A. As shown in FIG. 1B, device 200 comprise a PCR well 205 like PCR well 105 (FIG. 1A), with FIG. 1B further illustrating an overall volume of a polymerase chain reaction (PCR) mixture 240 present within the well 205. In some examples, the upper surface 242 of the overall volume of the PCR mixture 240 within PCR well 205 may comprise a flat meniscus as shown in FIG. 1B (or other) contour resulting from an example reduced-wetting portion 116 on the inner surface 114 of the side wall 110 of well 205. It will be understood that the flat meniscus shown in FIG. 1B is intended to be generally representative of other contours, such as a convex meniscus or minimally concave meniscus resulting from the example reduced-wetting portion 116 on the inner surface 114 of the side walls 110.

In some examples, the overall volume of the PCR mixture 240 as shown in FIG. 1B also may sometimes be referred to as exhibiting (or being shaped with) a generally uniform distribution within the well 205 at least in the sense that the upper surface 242 of the overall volume of the PCR mixture 240 has a height (F1) which is substantially the same across a width (W1) of the interior 125 of the PCR well 205. Similarly, in some examples in which the upper surface 242 of the overall volume of the PCR mixture 240 exhibits a generally flat meniscus, the upper surface 242 also may be described as being generally planar or having a generally flat shape.

In some examples, at least some of the desired effects from providing side walls having a reduced-wetting properties also may be achieved according to a contact angle which produces a minimally concave meniscus at least in the sense that a greater proportion of the overall volume of the PCR mixture 240 will be located in more central area of the PCR well 205 even though a flat or convex meniscus was not achieved. In some such examples, the minimal concavity meniscus (or generally uniform distribution) of the overall volume of the PCR mixture 240 may be expressed as the height (F1) of the upper surface 242 of the overall volume (of the PCR mixture 240) having a variance of less than about 10 percent (e.g. 9.5, 9.6, 9.7, 9.8, 9.9, 10, 10.1, 10.2, 10.3, 10.4, 10.5) within, and across a width of, the well 205. In some examples, the variance may comprise less than about 5 percent (e.g. 4.5, 4.6, 4.7, 4.8, 4.9, 5, 5.1, 5.2, 5.3, 5.4, 5.5).

In some examples, the reduced-wetting portion 116 of the side walls 110 of the PCR well 105, 205 may comprise a material(s) to cause the overall volume of the PCR mixture 240 to exhibit a contact angle of at least about 45 degrees (e.g. 44.5, 44.6, 44.7, 44.8, 44.9, 45, 45.1, 45.2, 45.3, 45.4, 45.5) relative to the inner surface 114 of side wall 110. In some examples, the contact angle may comprise at least about 50 degrees (e.g. 49.5, 49.6, 49.7, 49.8, 49.9, 50, 50.1, 50.2, 50.3, 50.4, 50.5), at least about 55 degrees (e.g. 54.5, 54.6, 54.7, 54.8, 54.9, 55, 55.1, 55.2, 55.3, 55.4, 55.5), at least about 60 degrees (e.g. 59.5, 59.6, 59.7, 59.8, 59.9, 60, 60.1, 60.2, 60.3, 60.4, 60.5), at least about 65 degrees (e.g. 64.5, 64.6, 64.7, 64.8, 64.9, 65, 65.1, 65.2, 65.3, 65.4, 65.5), at least 70 degrees (e.g. 69.5, 69.6, 69.7, 69.8, 69.9, 70, 70.1, 70.2, 70.3, 70.4, 70.5), or at least about 75 degrees (e.g. 74.5, 74.6, 74.7, 74.8, 74.9, 75, 75.1, 75.2, 75.3, 75.4, 75.5).

In general terms, the fluoropolymer component of the side wall 110 of the PCR well 205 may be incorporated in a variety of methods and/or expressed as a variety of different structures at inner surface 114 of the side walls. Some examples may comprise applying the fluoropolymer material as a liquid coating (e.g. dip, spray, etc.) on the polymer material of the side wall 110 and/or as part of a compounded mixture together with the polymer material for injection molding to form the at least one well (including formation of side walls 110). In some examples, the polymer material of the side wall 110 may comprise a cyclic olefin copolymer (COC) material. In some examples, the polymer material may comprise comprises polyethylene, polypropylene, polycarbonate, polymethylmethacrylate (PMMA), and the like.

Further details regarding various example materials suitable to form reduced-wetting portion 116, and/or the side wall 110 of the well (105, 205), are further described in association with at least FIGS. 4-6.

In some examples, the PCR mixture 240 includes components to execute three basic steps of a polymerase chain reaction, such as via pulse-controlled amplification, thermal cycling. Among other components, the PCR mixture 240 may comprise beads, primers, nucleic acid strands (e.g. DNA strands, RNA strands, portions thereof), probes, and deoxyribose nucleotides (dNTPs). A first step in thermal cycling may comprise denaturation in which the reaction volume is heated to about 94-98° C., which causes double-stranded DNA within the reaction mixture 240 to melt by breaking the hydrogen bonds between complementary bases, yielding two single-stranded DNA molecules. A second step in thermal cycling may comprise annealing in which less heat is applied to lower the reaction temperature to about 50-65° C., which allowing annealing of the primers to each of the single-stranded DNA templates as part of the reaction process. A third step in thermal cycling may comprise extension (i.e. elongation) in which the heat applied to the reaction volume is selected to create a reaction temperature suitable for the particular DNA polymerase used. In some examples, one target activity temperature for a thermostable DNA polymerase including Taq polymerase (e.g. a thermophilic eubacterial microorganism, Thermus aquaticus) is approximately 75-80° C. In this third step, the DNA polymerase synthesizes a new DNA strand complementary to the DNA template strand by adding free nucleoside triphosphates (dNTPs) from the reaction mixture. In some examples, the temperature used in these three phases of thermal cycling may vary depending on the length of the nucleic acid strand, the time available, the type of target (e.g. RNA, DNA, etc.), the density of polymerase and primers, etc. In some examples, the temperature used in these three phases of thermal cycling may vary depending on the length of the nucleic acid strand, the time available, the type of target (e.g. RNA, DNA, etc.), the density of polymerase and primers, etc.

It will be understood that in some examples such as reverse transcriptase PCR (RT-PCR) implementations, the second and third steps (annealing and extension) may be combined and operate at a single temperature of about 65° C. In some examples, such reverse transcriptase implementations may be performed via (or as) pulse-controlled amplification (PCA) type of polymerase chain reaction.

In some examples, the thermal cycle for a polymerase chain reaction (PCR), according to a pulse-controlled amplification, thermal cycling method, may be triggered by applying a current pulse of between about 20 Volts to about 60 Volts, and having a duration of about 0.3 to about 2 milliseconds. In some such examples, the current pulse may comprise about 40 Volts with a pulse duration of about 1 millisecond or other suitable parameters according to size of the PCR well 105, volume of the PCR mixture, the size, materials, shape of the first element 121 by which the heat is generated, etc. In some such examples, the current pulse may comprise on the order of 100 amps, such as 105 amps. It will be understood that the above-identified parameters may vary somewhat depending on a size of the PCR well 105, volume of the PCR mixture, and the size, materials, shape of the first element 121 (e.g. electrically resistive element) by which the heat is generated, etc.

In some examples, a zone in which the thermal cycling occurs may sometimes be referred to as a thermal cycling zone Z1 which is within (e.g. equal to or less than) a predetermined distance H1 (e.g. about 3, 4, or 5 micrometers) extending outward from the heat-generating, electrically resistive sheet 121 of the bottom 120 of the PCR well 105, 205. In some examples, this distance H1 may correspond to, and sometimes be referred to as, being within a close thermal proximity to the bottom. In some examples, the thermal cycling zone also may include a target thermal cycling zone where magnetic forces draw superparamagnetic beads to heighten the effectiveness of the pulse-controlled amplification of the PCR process.

In some examples, via the signal source (e.g. 170 in FIG. 1B) and the electrically resistive first element 121, the heat (H) is applied in controlled pulses in order to amplify (i.e. pulse-controlled amplification) reaction processes involving the polymerase chain reaction (PCR) mixture 240 within the thermal cycling zone Z1. In some such examples, the thermal cycling zone Z1 subject to a denaturation temperature (e.g. at least 90 degrees Celsius) comprises less than about 5 percent (e.g. 4.5, 4.6, 4.7, 4.8, 4.9, 5.0, 5.1, 5.2, 5.3, 5.4, 5.5) of the overall volume of the PCR mixture 240. In some examples, the thermal cycling zone Z1 subject to the denaturation temperature comprises less than about 4 percent (e.g. 3.5, 3.6, 3.7, 3.8, 3.9, 4.0, 4.1, 4.2, 4.3, 4.4, 4.5) of the overall volume of the PCR mixture 240, less than about 3 percent (e.g. 2.5, 2.6, 2.7, 2.8, 2.9, 3.0, 3.1, 3.2, 3.3, 3.4, 3.5) of the overall volume of the PCR mixture 240, less than about 2 percent (e.g. 1.5, 1.6, 1.7, 1.8, 1.9, 2.0, 2.1, 2.2, 2.3, 2.4, 2.5) of the overall volume of the PCR mixture 240, or less than about 1 percent (e.g. 0.5, 0.6, 0.7, 0.8, 0.9, 1.0, 1.1, 1.2, 1.3, 1.4, 1.5) of the overall volume of the PCR mixture 240.

As further shown in FIG. 1B, in some examples, the testing device 200 may comprise a magnet 172, which may be located external to the PCR well 205 in some examples. It will be understood that the magnet 172, as represented in FIG. 1B, may take a variety of shapes, sizes, etc. and is not limited by the appearance shown in FIG. 1B. In some examples, the magnet 172 applies an external magnetic force (arrows MF) at least to the overall volume of the PCR mixture 240 within the well 205, and in particular to the superparamagnetic beads 246 (functionalized with single-stranded nucleic acids) within the overall volume of the PCR mixture 240. It will be further understood that the arrows MF are representative of a magnetic force array extending generally across the entire thermal cycling zone Z1.

Via action of the magnetic forces (MF), the superparamagnetic beads 246 are attracted to (and drawn within) the thermal cycling zone Z1 to enhance the presence of the desired components (e.g. nucleic acid strands) of the PCR mixture to become subject to the pulse-controlled amplification in the thermal cycling zone Z1. By doing so, the PCR well 205 may facilitate testing which is more repeatable (e.g. more reliable) or more sensitive, such as being able to detect lower quantities (or concentrations) of a particular analyte of interest (e.g. virus, other).

FIG. 1B illustrates at least some example superparamagnetic beads 246 (functionalized with single-stranded nucleic acids) being present on or near inner surface 117A of bottom 120 of well 205. In this position, the superparamagnetic beads 246 are present within the thermal cycle zone (shown in dashed lines Z1) in which the pulse-controlled amplification takes place. Because of the reduced-wetting on inner surface 114 of side wall 110 caused by the portion 116, the overall volume of the PCR mixture 240 forms a target meniscus (e.g. flat, convex, minimal concavity) as represented by upper surface 242 such that a substantially greater proportion of the overall volume of the PCR mixture 240 becomes aligned with the thermal cycling zone Z1. In some examples, the term “substantially greater” may comprise at least 50 percent.

Via this arrangement, prior to application of an external magnetic force and/or prior to initiating the polymerase chain reaction, the superparamagnetic beads 246 (functionalized with single-stranded nucleic acids) within the overall volume of the PCR mixture 240 are already much closer to the target thermal cycling zone Z1 so that a much higher proportion (or absolute quantity) of beads 246 (functionalized with single-stranded nucleic acids) will be present within the thermal cycling zone Z1 upon initiation and execution of the polymerase chain reaction. Stated differently, in the absence of the reduced-wetting portion 116 (as represented by the dashed lines 260 corresponding to a highly concave meniscus), a much smaller proportion of the overall volume of the PCR mixture 240 would be present within the thermal cycling zone Z1, and significant portions of the overall volume of the PCR mixture 240 external to the zone Z1 would not be able to reach the thermal cycling zone Z1 soon enough to become part of the polymerase chain reaction. Therefore, in the absence of the example reduced-wetting portion 116 of side walls 110, the portion of the overall volume of the PCR mixture 240 to be exposed to the thermal cycling zone Z1 would be smaller than desired and result in underperformance of the pulse-controlled amplification, polymerase chain reaction.

In some examples, the device 200 may comprise an optical detector 174. In some such example, the optical detector 174 is aligned with, or alignable with, the openings 132 in resistive sheet 121 of the bottom 120 of the PCR well 205. The optical detector 174 is to receive light indicative of a quantity or volume of certain components within the well 205. In some examples, the optical detector 174 may optically detect the presence, quantity, etc. of fluorophores (F in FIG. 1B), which are an output of the pulse-controlled amplified, polymerase chain reaction (from PCR mixture 240) within well 205. Each fluorophore may correspond to an analyte of interest (virus particle, such as COVID 19, other) identified via the PCA-PCR reaction. As shown in FIG. 1B, the generally flat (or convex concave or minimally concave) meniscus resulting from the reduced-wetting portion 116 of side walls 110 may enable a greater proportion of such fluorophores (or other output element of the PCA-PCR) to migrate or diffuse into portions of the overall volume of the PCR mixture 240 which are in the path of optical detection. Via this arrangement, testing performed via PCR well 205 may result in significantly more effective capture of the actual output (e.g. quantity of fluorophores) of the PCA-PCR reaction within well 205 for a particular analyte of interest.

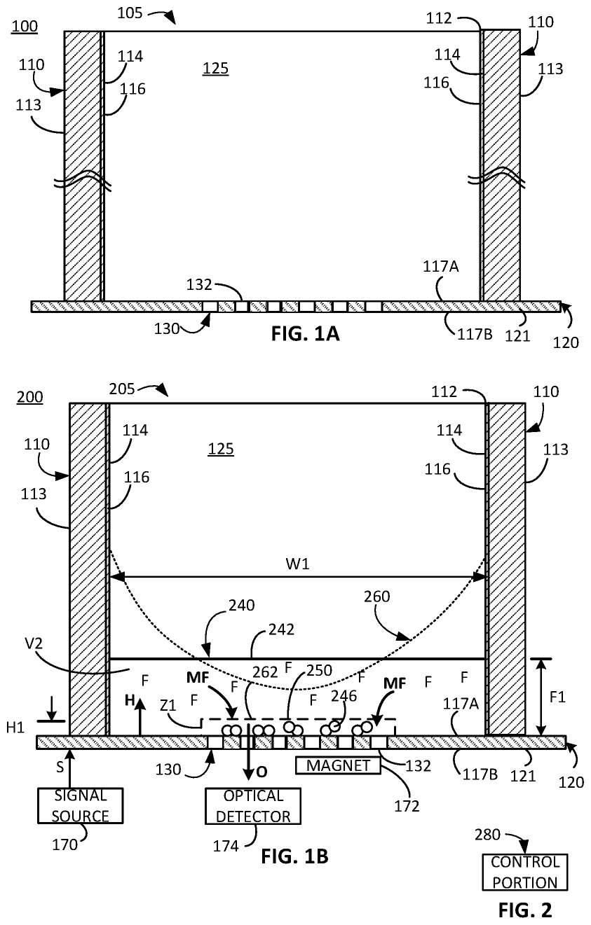

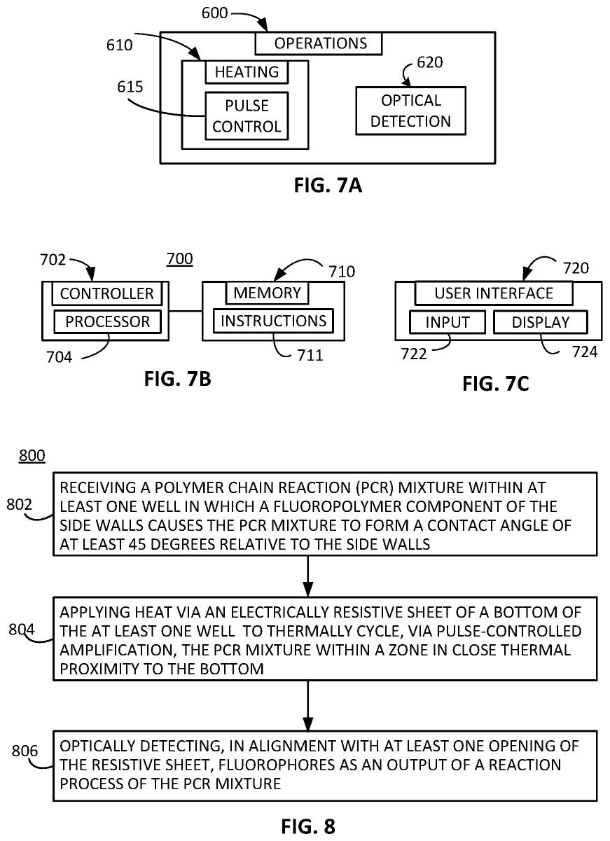

In some examples, at least some aspects of operation of, and/or monitoring of, the device 200 may be implemented via a control portion 280, as shown in FIG. 2. In some examples, the control portion 280 may comprise at least some of substantially the same features as, or comprise an example implementation of, the example control portion 700 in FIG. 7B.

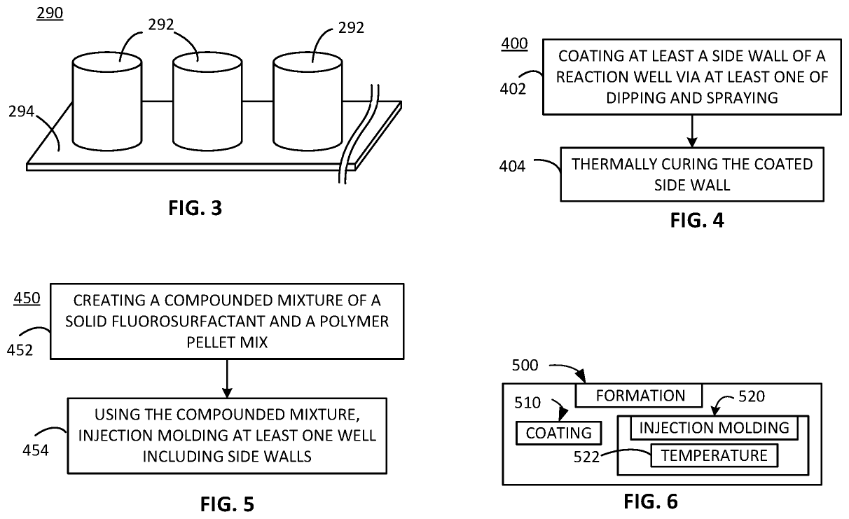

FIG. 3 is an isometric view schematically representing an example testing device 290 (e.g. molecular testing device) comprising a plurality of PCR wells 292 arranged on a common support 294. In some instances, the entire device may sometimes be referred to as a well plate. In some examples, at least some of the wells 292 comprise at least some of substantially the same features and attributes including (or related to) the wells (e.g. 105 in FIG. 1A, 205 in FIG. 1B) as described in association with at least FIGS. 1A-2 and 4-8. It will be understood that testing device 290 is not limited to the number (e.g. 3) of wells 292 shown in FIG. 3, such that device 290 may comprise a greater number or lesser number of wells 292. Moreover, in some examples, testing device 290 may comprise wells 292 arranged in a two-dimensional array (e.g. 2x2, 3x2, 4x2, etc.). In some examples, the support 294 and/or individual wells 292 may comprise a portion of, and/or be in communication with, control portion (e.g. 280 in FIG. 2; 700 in FIG. 7B). Furthermore, in some examples the testing device 290 also may be removably connectable to a console, station, or the like to support performing, monitoring, evaluating, etc. tests in the wells 292, with the respective console (or station, other) comprising at least a portion of (or incorporating) the control portion (e.g. 280 in FIG. 2; 700 in FIG. 7B).

FIG. 4 is a flow diagram schematically representing an example method 400 of coating a PCR well with the fluoropolymer component, such as a fluoropolymer liquid material. As shown in FIG. 4, method 400 comprises coating a side wall of a PCR well with a fluoropolymer liquid material (e.g. a solution) via at least one of dip coating and spray coating. It will be understood that, prior to coating, the side wall has been previously formed, such as via molding using a polymer material. After such coating processes, the coated well (e.g. 105, 205 in FIGS. 1A, 1B) may be cured. In some examples, such coating (e.g. dip, spray, etc.) is performed without plasma treatment of the side walls of the well (e.g. 105, 205).

In some working examples, the polymer material used to form the well, including the side wall, comprised a cyclic olefin copolymer (COC) material. Six specific examples of coating the side well with the fluoropolymer liquid material were tested. For these tests, the COC was dip-coated, but the examples also could have utilized spray coating. For these tests, sample molded articles of the polymer material (e.g. COC) were dip-coated in six different fluoropolymer coatings.

In one working example, the fluoropolymer material comprised a carbon-based fluoropolymer material. In some such examples, the fluoropolymer material comprised a C6 fluoro-carbon, such as but not limited to such materials obtainable from Cytonix® under the trade name Fluoropel 800 0.2%. This fluoropolymer material was converted into a liquid material via use of a solvent, such as but not limited to ethyl nonafluoroisobutyl ether and ethyl nonafluorobutyl ether. Other similar example fluoropolymer materials may be obtained from Cytonix® under the trade name Plastics 0P2. In another working example, the fluoropolymer material comprises a fluoroacrylic copolymer solution in a fluorosolvent, such as but not limited to such those obtainable from Cytonix® under the trade names TFM 100, TFM 100+.

In one working example, the fluoropolymer material was obtained from Aculon® of San Diego, California under the trade name E-FN to form a liquid solution via a solvent (e.g. methyl ethyl nonafluoroisobutyl ether and methyl nonafluorobutyl ether). In another working example, the fluoropolymer material was obtained from Aculon® under the trade name Nanoproof 5.0 was used to form a liquid solution material via a solvent, such as 3-ethoxy 1,1,1,2,3,4,4,5,5,6,6,6 docecafluoro-2-trifluoromethyl-hexane.

In some of the working examples, the example fluoropolymer materials obtained from Cytonix were coated according to an example recipe in which molded shaped articles (e.g. thin rectangular shaped members) were dipped twice at a controlled down speed (e.g. 300,000 micrometers/minute) and controlled up speed (e.g. 600,000 micrometers/minute), with a lower hold time of about 20 seconds.

In some of the working examples, the example fluoropolymer materials obtained from Aculon were coated according to an example recipe in which molded shaped articles (e.g. thin rectangular shaped members having a thickness of 1 millimeter) were dipped once at a controlled down speed (e.g. 300,000 micrometers/minute) and controlled up speed (e.g. 100,000 micrometers/minute), with a lower hold time of about 20 seconds.

In all of these working examples, the dipped molded articles were thermally cured at 80 degrees C. for 10 min. However, these articles could have been be cured at room temperature or at other temperatures.

Upon completion of the curing, each example coated article was exposed to a PCR mixture and, thereafter measurements were obtained of the contact angle between the PCR mixture and the example dip-coated articles. For reference purposes, an uncoated article made of the cyclic olefin copolymer (COC) exhibited a contact angle of about 20 degrees and at which substantial wetting occurs, which would be expected to (and was observed to) result in a significant concave meniscus when this arrangement was deployed on side walls of a PCR well.

On the other hand, all of the above-noted working examples in which the cyclic olefin copolymer (COC) material was coated with a fluoropolymer solution, a contact angle of about 85 degrees was observed between the coated molded article and the PCR mixture. Accordingly, each of the working coating examples resulted in about a 65 degree increase in the contact angle of the PCR mixture relative to the coated, molded article as compared to the observed contact angle of 20 degrees for an uncoated, molded article.

Accordingly, the working examples demonstrated that a fluoropolymer coating on a molded article, which would be used to form PCR wells should provide reduced wetting and a corresponding meniscus which is convex, flat, or of minimal concavity.

FIG. 5 is a flow diagram schematically representing an example method 450 of forming side wells of a PCR well to exhibit a reduced-wetting portion 116 (e.g. FIGS. 1A-1B). In general terms, a PCR well or array of PCR wells on a well plate may be formed via injection molding using a polymer material. In this example method 450, a fluorosurfactant is introduced to modify the wetting properties (e.g. contact angle) of PCR mixture relative to the side wall (e.g. 110), such as the wetting properties described in association with at least FIGS. 1A-3.

As shown in FIG. 5, the example method 450 comprises creating a compounded mixture of a solid fluorosurfactant and a polymer pellet mix. As further shown at 454 in FIG. 5, using the compounded mixture, method 450 further comprises injection molding at least one well including side walls.

In some such examples, the fluorosurfactant is incompatible the polymer material such that the fluorosurfactant is expected to bloom or migrate to the surface of the polymer material both during and after the injection molding process. As a result of such blooming and/or migration, an inner surface 114 (FIGS. 1A, 1B) of the side walls 110 is expected to develop portion 116 including the fluoropolymer surfactant to provide the intended reduced-wetting properties (e.g. non-wetting, minimal wetting, etc.) as expressed via the example contact angles (e.g. at least 45 degrees, 50 degrees, etc.), as previously described in association with at least FIGS. 1A-3.

Accordingly, in some examples, the injection molding per method 450 is performed within a temperature range protective of the fluorosurfactant additive. In particular, in order to perform injection molding of a PCR well using some example polymer materials, at least some heating phases (e.g. within a barrel of an injection molding device) may involve temperatures exceeding a limit of heating for the fluorosurfactant additive. For instance, when the polymer material comprises a cyclic olefin copolymer, the injection molding process may include heating the polymer material to temperatures exceeding 230 degrees Celsius (C). However, many dry melt fluorosurfactant additives cannot be heated above 250-280C while still maintaining an integrity, properties, etc. of those additives. Accordingly, some example methods may control the heating phases of the injection molding process to ensure that the maximum temperature does not exceed the heating limit of the fluorosurfactant additive material.

One example solid fluorosurfactant comprises a TFMA-6 surfactant material available from Cytonix, LLC of Beltsville, Maryland. One prophetic example procedure may comprise adding 0.5-2% (preferably 1% by weight) of the fluorosurfactant additive material into the polymer material (e.g. COC) pellet mix and then performing the injection molding process as noted above. Another prophetic example procedure may comprise first making a masterbatch of concentrated TFMA-6 in the polymer material (e.g. COC) at a 12.5% by weight loading, and then using this masterbatch mixture in the injection molding process to achieve a final concentration of TFMA-6 as noted above.

In some examples, forming the reduced-wetting portion 116 of side walls 110 may be performed via adding a fluorosurfactant (as in the above-described examples) but without also adding a separate or second fluoropolymer material.

FIG. 6 is a block diagram schematically representing an example formation engine 500. In general terms, the formation engine 500 enables tracking and/or controlling formation of a side wall of a PCR well to exhibit reduced-wetting properties (e.g. non-wetting, minimal wetting, etc.) of the side wall relative to an overall volume of a PCR mixture, as previously described in association with at least FIGS. 1A-5. As shown in FIG. 6, in some examples the formation engine 500 may comprise a coating engine 510 and an injection molding engine 520 including a temperature parameter 522. In some examples, the coating engine 510 is to track and/or control a coating process, such as but not limited to, the example method 400 of FIG. 4.

In some examples, the injection molding engine 520 is to track and/or control an injection molding process, such as but not limited to, the example method 450 of FIG. 5. In some such examples, the temperature parameter 522 is to generally track and/or control the temperature during the injection molding process. In one aspect, this tracking and/or control acts to ensure that the heating limit of a solid surfactant additive material is not exceeded to preserve the integrity, properties, etc. of that additive material.

FIG. 7A is a block diagram schematically representing an example operations engine 600. In some examples, the operations engine 600 may form part of a control portion 700, as later described in association with at least FIG. 7B, such as but not limited to comprising at least part of the instructions 711. In some examples, the operations engine 600 may be used to implement at least some of the various example devices and/or example methods of the present disclosure as previously described in association with FIGS. 1A-6 and/or as later described in association with FIGS. 7B-8. In some examples, the operations engine 600 (FIG. 7A) and/or control portion 700 (FIG. 7B) may form part of, and/or be in communication with, a testing device (including at least one PCR well) such as the devices and methods described in association with at least FIGS. 1A-6 and 7C-8.

In some examples and in general terms, the operations engine 600 directs, monitors, and/or reports information regarding a polymerase chain reaction (PCR) to occur within at least one well of a testing device, with the polymerase chain reaction (PCR) comprising a pulse-controlled amplification (PCA) type of polymerase chain reaction, in some examples. As shown in FIG. 7A, in some examples the operations engine 600 may comprise a heating engine 610 and an optical detection engine 620. The heating engine 610 may track and/or control heating, such as generated via the electrically resistive sheet (e.g. 121 in FIGS. 1A-1B). In some such examples, the heating engine 610 may track and/or control the heating according to a pulse-controlled amplification (PCA) parameter 615 to perform the polymerase chain reaction (PCR) within the PCR well (e.g. 105, 205) via pulse-controlled amplification.

In some examples, the optical detection engine 620 may track and/or control optical detection of aspects of a polymerase chain reaction within a PCR well (e.g. 105, 205), such as but not limited to, optical detection of fluorophores as an output of the polymerase chain reaction. In some such examples, a volume or quantity of the detected fluorophores may be indicative of a presence, intensity, prevalence, etc. of a particular virus within the sample associated with the reaction mixture deposited within the well (e.g. 105, 205). In some examples, the optical detection engine 620 implements the optical detection via optical detector (e. g. 174 in FIG. 1B). In some examples, the optical detector may detect a fluorophore signal intensity.

It will be understood that various engines and parameters of operations engine 600 may be operated interdependently and/or in coordination with each other, in at least some examples.

FIG. 7B is a block diagram schematically representing an example control portion 700. In some examples, control portion 700 provides one example implementation of a control portion forming a part of, implementing, and/or generally managing the example testing devices (e.g. molecular testing devices), as well as the particular portions, components, wells, side wall coatings or structures, molding, signal sources, electrically resistive sheets, magnets, optical detectors, operations, control portion, instructions, engines, functions, parameters, and/or methods, as described throughout examples of the present disclosure in association with FIGS. 1-7A and 7C-8. In some examples, control portion 700 includes a controller 702 and a memory 710. In general terms, controller 702 of control portion 700 comprises at least one processor 704 and associated memories. The controller 702 is electrically couplable to, and in communication with, memory 710 to generate control signals to direct operation of at least some of the example molecular testing devices, as well as the particular portions, components, wells, side wall coatings or structures, molding, signal sources, electrically resistive sheets, magnets, optical detectors, operations, control portion, instructions, engines, functions, parameters, and/or methods, as described throughout examples of the present disclosure. In some examples, these generated control signals include, but are not limited to, employing instructions 711 stored in memory 710 to at least direct and manage testing operations via examples of the present disclosure. In some instances, the controller 702 or control portion 700 may sometimes be referred to as being programmed to perform the above-identified actions, functions, etc.

In response to or based upon commands received via a user interface (e.g. user interface 720 in FIG. 7C) and/or via machine readable instructions, controller 702 generates control signals as described above in accordance with at least some of the examples of the present disclosure. In some examples, controller 702 is embodied in a general purpose computing device while in some examples, controller 702 is incorporated into or associated with at least some of the example molecular testing devices, as well as the particular portions, components, wells, side wall coatings or structures, molding, signal sources, electrically resistive sheets, magnets, optical detectors, operations, control portion, instructions, engines, functions, parameters, and/or methods, etc. as described throughout examples of the present disclosure.

For purposes of this application, in reference to the controller 702, the term “processor” shall mean a presently developed or future developed processor (or processing resources) that executes machine readable instructions contained in a memory or that includes circuitry to perform computations. In some examples, execution of the machine readable instructions, such as those provided via memory 710 of control portion 700 cause the processor to perform the above-identified actions, such as operating controller 702 to implement testing operations via the various example implementations as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 710. The machine readable instructions may include a sequence of instructions, a processor-executable machine learning model, or the like. In some examples, memory 710 comprises a computer readable tangible medium providing non-volatile storage of the machine readable instructions executable by a process of controller 702. In some examples, the computer readable tangible medium may sometimes be referred to as, and/or comprise at least a portion of, a computer program product. In other examples, hard wired circuitry may be used in place of or in combination with machine readable instructions to implement the functions described. For example, controller 702 may be embodied as part of at least one application-specific integrated circuit (ASIC), at least one field-programmable gate array (FPGA), and/or the like. In at least some examples, the controller 702 is not limited to any specific combination of hardware circuitry and machine readable instructions, nor limited to any particular source for the machine readable instructions executed by the controller 702.

In some examples, control portion 700 may be entirely implemented within or by a stand-alone device.

In some examples, the control portion 700 may be partially implemented in one of the example testing devices and partially implemented in a computing resource separate from, and independent of, the example testing devices but in communication with the example testing devices. For instance, in some examples control portion 700 may be implemented via a server accessible via the cloud and/or other network pathways. In some examples, the control portion 700 may be distributed or apportioned among multiple devices or resources such as among a server, a testing device, a user interface.

In some examples, control portion 700 includes, and/or is in communication with, a user interface 720 as shown in FIG. 7C. In some examples, user interface 720 comprises a user interface or other display that provides for the simultaneous display, activation, and/or operation of at least some of the example testing devices, as well as the particular portions, components, wells, side wall coatings or structures, molding, signal sources, electrically resistive sheets, magnets, optical detectors, operations, control portion, instructions, engines, functions, parameters, and/or methods, etc., as described in association with FIGS. 1A-7B and 8. In some examples, at least some portions or aspects of the user interface 720 are provided via a graphical user interface (GUI), and may comprise a display 724 and input 722.

FIG. 8 is a flow diagram of an example method 800. In some examples, method 800 may be performed via at least some of the testing devices, as well as the particular portions, components, wells, side wall coatings or structures, molding, signal sources, electrically resistive sheets, magnets, optical detectors, operations, control portions, engines, functions, parameters, and/or methods, etc. as previously described in association with at least FIGS. 1A-7C. In some examples, method 800 may be performed via at least some testing devices, as well as the particular portions, components, wells, side wall coatings or structures, molding, signal sources, electrically resistive sheets, magnets, optical detectors, operations, control portions, engines, functions, parameters, and/or methods, etc. other than those previously described in association with at least FIGS. 1A-7C.

As shown at 802 in FIG. 8, in some examples method 800 comprises receiving a polymer chain reaction (PCR) mixture within at least one well in which a fluoropolymer component of the side walls causes the PCR mixture to form a contact angle of at least 45 degrees relative to the side walls. As further shown at 804 in FIG. 8, in some examples method 800 comprises applying heat via an electrically resistive sheet of a bottom of the at least one well to thermally cycle, via pulse-controlled amplification, the PCR mixture within a zone in close thermal proximity to the bottom of the at least one well. As further shown at 806 in FIG. 8, in some examples method 800 comprises optically detecting, in alignment with at least one opening of the resistive sheet, fluorophores as an output of the PCR mixture.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A device comprising:
at least one well to receive a polymerase chain reaction (PCR) mixture and including:
a bottom comprising an electrically resistive sheet including at least one opening and to receive a signal from a signal source to generate heat to cause a pulse-controlled amplification, thermal cycling zone in close thermal proximity to the resistive sheet; and
side walls comprising a polymer material and a fluoropolymer component to cause the PCR mixture to form a contact angle of at least 45 degrees relative to the side walls; and
an optical detector alignable with the at least one opening to detect fluorophores as an output of the PCR mixture.

2. The device of claim 1, wherein via the pulse-controlled amplification, within the at least one well, the thermal cycling zone subject to a denaturation temperature comprises less than about 5 percent of an overall volume of the PCR mixture.

3. The device of claim 1, wherein the contact angle is greater than 45 degrees to cause an overall volume of the PCR mixture to exhibit a generally uniform height profile within the at least one well in which a height of the overall volume exhibits a variance of less than 5 percent across a diameter of the at least one well.

4. The device of claim 1, wherein the polymer material comprises cyclic olefin copolymer (COC).

5. The device of claim 1, wherein the fluoropolymer component comprises a carbon-based fluoropolymer material.

6. The device of claim 3, wherein the fluoropolymer component further comprises a fluoroacrylic copolymer solution in a fluorosolvent.

7. The device of claim 1, wherein the side walls comprise an injection-molded structure of a compounded mixture comprising a polymer solids material and at least one of a fluoropolymer material and a fluorosurfactant material.

8. The device of claim 1, comprising:
an external magnet aligned to apply a magnetic force to superparamagnetic beads, functionalized with single-stranded nucleic acids, within the PCR mixture to draw the beads toward, and into, the thermal cycling zone.

9. A device comprising:
at least one well to receive a polymer chain reaction (PCR) mixture and comprising:
a bottom comprising an electrically resistive sheet including at least one opening and to receive a signal from a signal source to generate heat for pulse-control amplification, within the at least one well, in a thermal cycling zone in close thermal proximity to the resistive sheet and subject to a denaturation temperature less than 5 percent of an overall volume of the PCR mixture; and
side walls comprising a polymer material and a fluoropolymer component to cause the PCR mixture to form at least one of a convex meniscus and a flat meniscus; and
an optical detector alignable with the at least one opening of the resistive sheet to detect fluorophores as an output of the PCR mixture.

10. The method of claim 9, wherein the side walls comprise at least one of:
a molded polymer material coated with the fluoropolymer component; and
an injection-molded structure of a compounded mixture comprising a polymer solids material and at least one of a fluoropolymer material and a fluorosurfactant material.

11. A method comprising:
receiving a volume of a polymer chain reaction (PCR) mixture within at least one well in which a fluoropolymer component of side walls of the at least one well causes an overall volume of the PCR mixture to form a contact angle relative to the side walls to cause the overall volume to exhibit a generally uniform distribution across a diameter of an interior of the at least one well;
applying heat, via an electrically resistive sheet of a bottom of the at least one well, to thermally cycle, via pulse-controlled amplification, the PCR mixture within a zone in close thermal proximity to the resistive sheet; and
optically detecting, in alignment with an opening of the resistive sheet, fluorophores as an output of the reaction mixture.

12. The method of claim 11, comprising at least one of:
the thermal cycling zone subject to a denaturation temperature of less than about 5 percent of an overall volume of the PCR mixture; and
applying an external magnetic force to superparamagnetic beads, functionalized with single-stranded nucleic acids, within the PCR mixture within the at least one well to draw the beads toward, and into, the thermal cycling zone.

13. The method of claim 11, wherein the contact angle is at least 60 degrees.

14. The method of claim 11, wherein via the fluoropolymer component, the contact angle is to cause the PCR mixture to exhibit the substantially uniform distribution, within the at least one well, as at least one of a flat meniscus and a convex meniscus.

15. The method of claim 11, comprising:
forming the side walls to comprise a cyclic olefin copolymer (COC) material and including the fluoropolymer component as at least one of:
a coating on the COC material; and
part of a compounded mixture together with the COC material injection-molded to form the at least one well.
